(19) United States
(12) Patent Application Publication
(10) Pub. No.: US 2019/0000665 A1
(43) Pub. Date: Jan. 3, 2019

(54) THERMAL WRAP ARRANGEMENT FOR APPLYING HEAT/COLD TO THE MANDIBULAR REGION OF THE HEA

(71) Applicant: Rebecca America Dow, Wilton, NY (US)

(72) Inventor: Rebecca America Dow, Wilton, NY (US)

(21) Appl. No.: 16/008,466

(22) Filed: Jun. 14, 2018

Related U.S. Application Data
(63) Continuation-in-part of application No. 13/346,670, filed on Jan. 9, 2012.
Application No. 13/346,670 is a continuation-in-part of application No. 12/003,495, filed on Dec. 26, 2007, now abandoned.
Application No. 12/003,495 is a continuation-in-part of application No. 11/444,390, filed on Jun. 1, 2006, now abandoned.

(51) Int. Cl. A61F 7/02

(52) U.S. Cl. CPC A61F 7/02; A61F 2007/0013

(57) ABSTRACT

A thermal wrap having identical side portions, each having a pocket formed therein for receiving thermal material such as gel packs or instant/single use chemically activated thermal packs. The side portions are made of an expandable material and connected at the top by at least two independently attached non-elasticated side straps or loop straps which slide through buckles and attach to two adjacent hook pieces, and at the bottom by a pair of cooperating chin straps made from the same material as the main structure that connects the two pouches which further ensures that the pockets allow for even application as well as increased pressure of thermal energy to the head, neck, or face including the mandibular region of the face according to the users desirable fit and pressure. The pockets are sized to receive standard sized gel packs with dimensions relative to the region of the body being treated.

### CROSS REFERENCE TO RELATED APPLICATIONS

This application is a continuation in part of application Ser. No. 13/346,670, filed on Jan. 9, 2012, which is a continuation in part of application Ser. No. 12/003,495, filed on Dec. 26, 2007, which is a continuation in part of application Ser. No. 11/444,390, filed on Jun. 1, 2006.

### BACKGROUND OF THE INVENTION

### Field of the Invention

The present invention concerns therapeutic thermal treatment devices. More particularly, the invention is directed to a thermal treatment wrap configured for application of heat/cold to various parts of a human body, including the head, neck, and/or face, particularly the lower mandibular region.

### Description of the Prior Art

Thermal treatment wraps and the like are well known in the art. They typically consist of a web or sheet material which may be textile, elastic, or plastic; configured or configurable to cover a particular body part. They are typically conceived as one size fits all, or, in the alternative, limited to two or three sizes. While these arrangements are generally satisfactory, they can be ineffective when used to treat specific areas of the head, face, and neck. This is primarily because the human head, unlike other body parts, changes drastically in both size and contour from one individual to the next, and allowances have to be made for the bulk of the hair, as well as particular hairstyles. To that end, the typical therapeutic thermal head wrap has adjustable or elasticated straps, and one or more pockets or voids for containing thermal material such as gel packs, ice, etc. The drawback with these types of arrangements is that it is difficult to maintain effective thermal contact with a given treatment area. This problem is exacerbated by the use of gel packs which are dense, heavy, and inflexible not particularly conforming to contoured surfaces, the result being that the gel pack will either sag, pucker or not conform to the area to be treated, and therefore be only marginally effective at a given treatment area. Prior art devices typically attempt to solve the problem by providing an elasticated material within which a gel pack is contained. The solution is only temporary at best, as the gel packs are heavy, inflexible and can deform the material over time irreversibly rendering it ineffective to accurately position the gel pack. Another problem with prior art devices is that the device will typically have a single large pocket for containing a gel pack, the pocket not conforming to any of the standard gel pack sizes. Another problem with existing therapeutic wraps and bandages is the use of elasticated material for much if not all of the wrap. This material, over time, stretches out of shape. The new stretched out of shape material does not allow for positioning the gel pack to affect the desired treatment area.

U.S. Pat. No. 5,837,004 issued to Lavore is typical of the head and face thermal treatment accessories. The device has only one, hexagonal shape pouch, for containing a thermal material of irregular size, the pocket connected at opposite ends by a single adjustable strap. The device is one size fits all. By contrast, the present invention is directed to a thermal compress arrangement having identical side portions connected at one end by a pair of releasably attached tension straps set wide enough apart to prevent slipping from the user's head, face or neck, and at the other end by a releasably attached chin strap. It can be appreciated that the releasably attached two side tension straps in the present invention can apply even pressure to the thermal material in either pocket as they are set-apart wide enough to secure the thermal material within the pouch and hold the gel pack to the treatment area without shifting.

U.S. Pat. No. 5,188,103 issued to Smith discloses a simple elastic band having a repositionable thermal pouch. The band will eventually stretch to a point that it cannot be held in place about the user [symbol] s head at which point it must be replaced. The present invention contemplates a thermal treatment wrap designed for treatment of the mandibular region which has pockets sized in accordance with standard gel pack sizes. The wrap 10 can be available in a range of sizes, with the pocket size selected in accordance with both the standard gel pack sizes according to the area being treated.

The present invention is directed to a thermal wrap having identical side portions, each having a pocket formed therein for receiving thermal material such as gel packs or instant/single use chemically activated thermal packs. The side portions are made of an expandable material and connected at the top by at least two independently attached non-elasticated side straps or loop straps which slide through buckles and attach to two adjacent hook pieces, and at the bottom by a pair of cooperating chin straps made from the same material as the main structure that connects the two pouches which further ensures that the pockets allow for even application as well as increased pressure of thermal energy to the head, neck, or face including the mandibular region of the face according to the users desirable fit and pressure. The pockets are sized to receive standard sized gel packs with dimensions relative to the region of the body being treated.

### SUMMARY OF THE INVENTION

It is a major object of the invention to provide an improved thermal wrap.

It is another object of the invention to provide an improved thermal wrap particularly adapted for use on a human head, face and neck.

It is another object of the invention to provide an improved thermal wrap for treatment of the mandibular region of a human head.

It is another object of the invention to provide an improved, more secured pouch closure that ensures that the gel packs will stay in place while the wrap is being applied and in position, adjustable thermal wrap for treatment of the mandibular region of a human head, and the range of shapes and sizes of the human head, and the neck.

It is a major object of the invention to provide an improved adjustable thermal wrap where the use of a tensioning strap system reduces stretching, slipping and twisting on the targeted region and permanent displacement of the thermal material of the wrap.

It is yet another object of the invention to provide an improved thermal wrap with means for receiving standard sized thermal treatment packs.

Finally, it is a general goal of the invention to provide improved elements and arrangements thereof in an apparatus for the purposes described which is dependable and fully effective in accomplishing its intended purposes.

These and other objects of the present invention will become readily apparent upon further review of the following specification and drawings.

The present invention meets or exceeds all the above objects and goals. Upon further study of the specification and appended claims, further objects and advantages of this invention will become apparent to those skilled in the art.

### BRIEF DESCRIPTION OF THE DRAWINGS

Various other objects, features, and attendant advantages of the present invention will become more fully appreciated as the same becomes better understood when considered with the accompanying drawings, in which like reference characters designate the same or similar parts throughout the several views, and wherein:

FIG. 1 shows a front perspective view of the thermal wrap of the invention positioned on a user's face.

FIG. 2 shows a perspective view of one side of the thermal wrap with gel pack inserted.

FIG. 3 shows a perspective view of the opposing side of the thermal wrap with gel pack inserted.

FIG. 4 shows a plan view of the outer side of the thermal wrap.

FIG. 5 shows a plan view of the inner side of the thermal wrap.

FIG. 6 shows a front perspective view of the thermal wrap of the invention positioned about the upper part of the user's head.

FIG. 7 shows a side view of the thermal wrap positioned as in FIG. 6.

FIG. 8 shows the opposite side view of the thermal wrap positioned as in FIG. 6.

FIG. 9 shows a rear view of the thermal wrap positioned as in FIG. 6.

FIG. 10 shows a side view of the thermal wrap positioned about the neck of the user.

FIG. 11 shows the opposite side view of the thermal wrap positioned as in FIG. 10.

FIG. 12 shows a rear view of the thermal wrap positioned as in FIG. 10 with gel pack inserted.

FIG. 13 shows a detail of the thermal wrap showing the pocket closure.

FIG. 14 illustrates an aspect of the tensioning procedure of the thermal wrap.

### DETAILED DESCRIPTION OF THE PREFERRED EMBODIMENTS

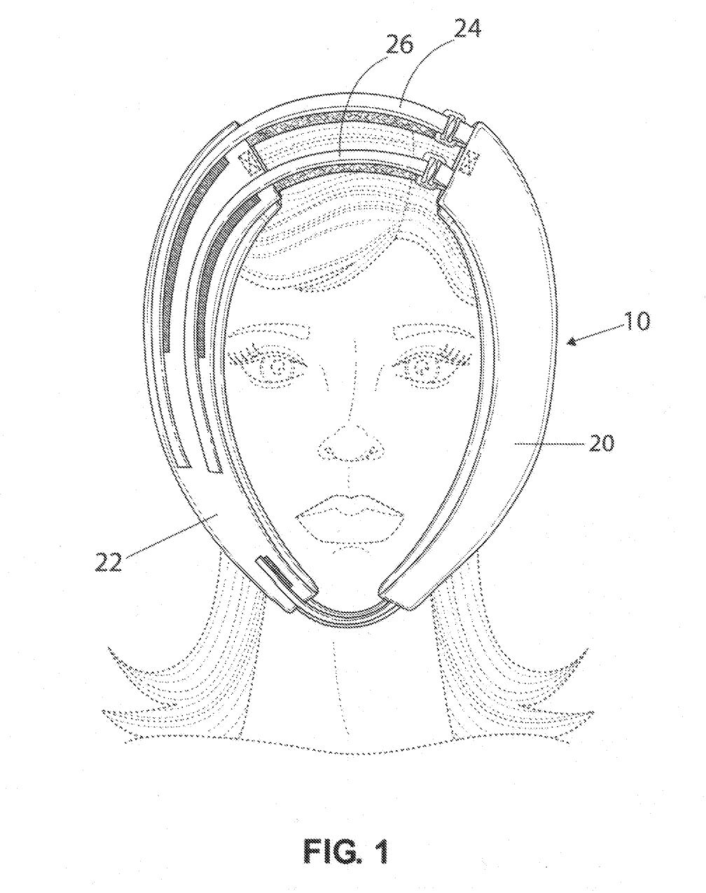

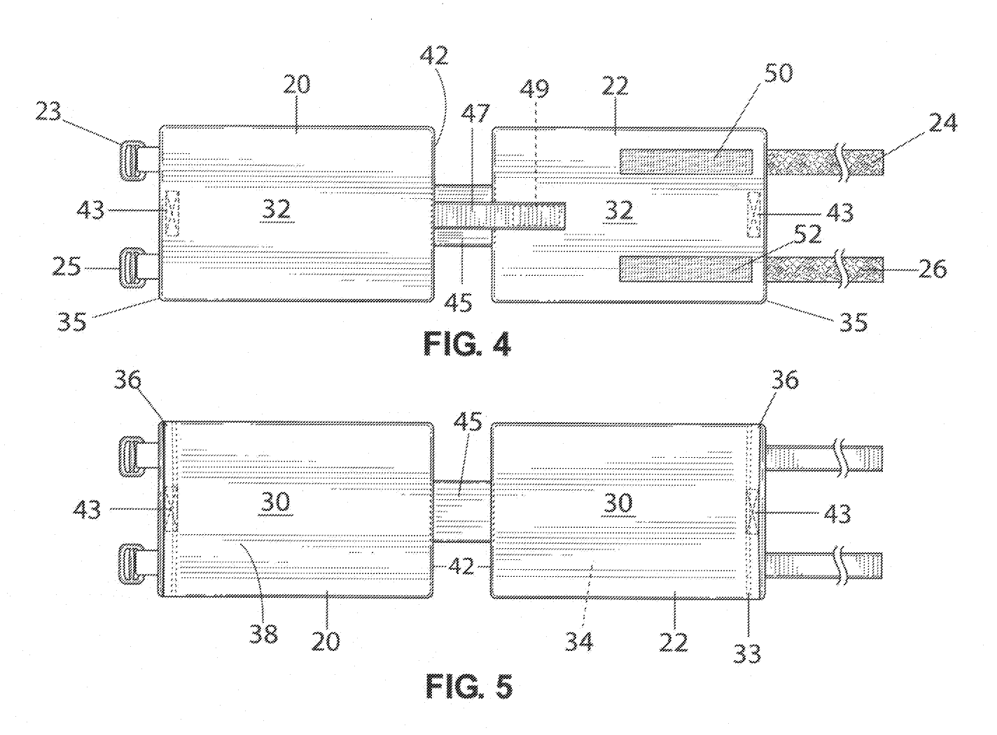

Referring now to FIGS. 1-5, the thermal wrap arrangement of the present invention, generally indicated by the numeral 10, is shown. It can be seen that the wrap 10 has a main body formed of two nearly identical side portions 20, 22, releasably connected at one end by a pair of straps 24, 26 and corresponding pair of buckles 23, 25. Straps 24, 26, are evenly spaced about the center of opposing ends of the side portion 22, and are preferably connected thereto by stitching. Buckles 23, 25 are attached to a short length of straps 27, 29 formed of e.g. woven elastic, sometimes termed “webbing”, which straps are attached by e.g. stitching to the top edge of pocket 20 to allow for adjustment as would be apparent to one of skill in the art. The straps 27, 29 have some elasticity whereas straps 24, 26 are of a standard loop side of a hook and loop fastener or Velcro® material and have limited elasticity to limit the possibility of these straps becoming permanently “stretched out” or elongated. Straps 24, 26 may be looped through one side of the respective buckle 23, 25 and tension can be adjusted as desired, then secured by pressing the appropriate length of strap 24, 26 onto side portion 22 as will be described in more detail below.

It should be noted that while the side portions 20, 22 are shown as rectangular, they may have any elongated or widened shape which can accommodate a side pocket which is sized to accept standard sized gel packs. The wrap 10 may be made available in incrementally increasing sizes starting from an extra small head (circumference) of approx. 18 inches or child [symbol] s head up to a maximum of about 26 inches. Of course the length of the straps 24, 26 may be increased or decreased to allow for a desired amount of adjustability. In an alternative one size fits all embodiment, the side portions 20, 22 may be an intermediate size, while the straps 24, 26 are elongated to allow for adjustment from the smallest to the largest head size. The even spacing and distance between the two buckles 23,25 and the independent side loop straps 24, 26, and their adjacent hook pieces 50,52, respectively, help reduce slipping of the device, and twisting of the wrap 10 due to space between the two independent tensioning areas 23,24,25 and 25,26,52 is wide enough to accommodate an extra-large head shape and size, and also, small enough where it can curve around an extra small head (3 year old or even smaller). It can be appreciated that more than two straps 24, 26 and their respective tensioning components 23,25 and 50,52 may be employed, especially for larger sizes of the wrap.

In a preferred embodiment, the wrap 10 is made primarily from a polyester or polyester/spandex, or nylon/spandex material or any synthetic material which has a slight elasticity, except as otherwise noted herein. Also, the material is preferably treated with anti-microbial agents, and is woven to be quick drying. A material such as recycled polyester may be ideally used for this purpose. Alternatively, the wrap 10 may be made from terrycloth, bamboo, cotton, nylon, or other synthetic or natural material but less expandable material, with some loss of conformance to the user [symbol] s head shape. Therefore a combination of spandex or lycra with any of the aforementioned materials could also be substituted for the material.

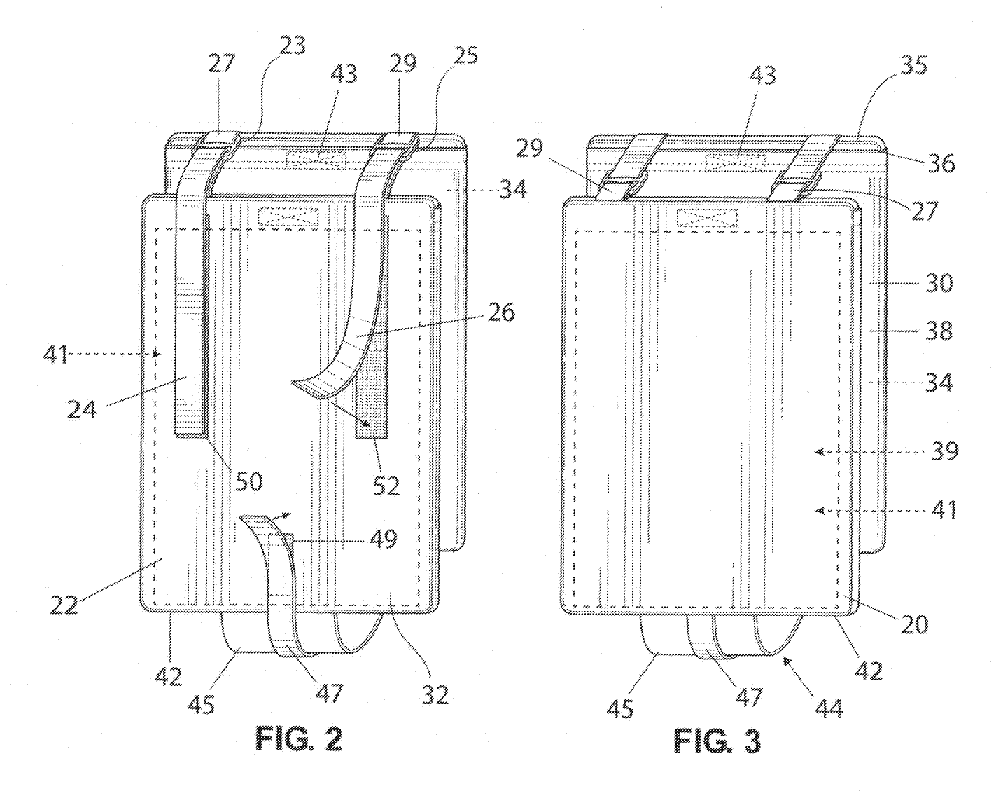

Referring specifically to FIGS. 2 and 3, it can be seen that each side portion 20, 22 has both inner and outer sheets 30, 32 which are unattached along their respective top edges 36, 35 and thereby form a pocket 38 into which a gel pack 39 may be inserted. An additional inner sheet 34 is attached to inner sheet 30 and is essentially a thermal transfer sheet as the gel pack 39 radiates thermal energy therethrough. The additional inner sheet 34 helps to limit the amount of thermal energy transferred to the user to prevent frostbite or other injuries which may occur as a result of excessive cold or heat applied to the user's skin. Thicker material may be used for inner and outer sheets 30, 32 in which case a single layer of material may be used for inner sheet 30 thereby eliminating the need for 34. The attachment of sheets 30, 32 is preferably secured by stitching, with the top, side, and bottom edges of the inner and outer sheets 30, 32 are bound together on the inside by stitching and further secured by a second stitch to enhance durability. The top edge 36 of inner sheet 30 is preferably folded over and securely stitched to thermal transfer sheet 34 as by stitches 33. If only one thicker material is used for inner sheet 30 so that 34 is not required to protect user from frostbite and/or injury from thermal transfer, the same coverstitching and/or straight stitching will be located at the top edge 36 of the inner sheet to clean finish the raw edge of the pocket bags. Although side pockets 38 may be wide enough so that the gel pack 39 may be slid horizontally, as well as tall enough to slide in different size gel packs or thermal compresses to adjust the region to which thermal energy is transferred, in a preferred embodiment the gel pack 39 is sized to fill the entire pocket 38.

The bottom edges 42 of the side pocket envelopes are connected by a chin strap portion 44, which is formed of primary 45 and secondary 47 straps. The primary chin strap 45 is relatively wide to ensure that inner and outer sheets 30, 32 are stretched taut about the mandibular region of the user's face, and is preferably about half the width of the side pockets 38. The primary chin strap 45 is made of the same material as the bulk of the wrap 10, namely, a polyester, nylon, or other fabric that can have a stretch material namely spandex or lycra. The secondary chin strap 47 is attached centrally (widthwise) to the bottom edge 42 of side portion 20 and is relatively narrow, about a third of the width of the primary chin strap 45, and centrally positioned relative to the position of straps 24, 26. The secondary strap 47 is made from a strip of Velcro® loop material or a generic hook and loop fastener, specifically the loop side of the hook and loop fastener, and is therefore not elasticated. The sizing of the chin strap portion 44 relative to the width of the pockets 38 overcomes a tendency common in prior art thermal wraps where bunching or other fabric deformations due to a single, relatively narrow (widthwise) strap lead to an uneven application of the thermal treatment. Also, prior art wraps having a chin strap substantially as wide as the pockets will have a tendency to cause deformation or sagging of the pocket material when the gel pack or compress is inserted which is rectified by the added secondary tension strap 47, when placed around the user's head, neck or face. The positioning of the chin strap portion 44 in the present inventive wrap 10 allows for tensioning of the central part of the side portions 20, 22 in the lengthwise direction.

Secondary chin strap 47 is attached by stitching to the lower edge 42 of side portion 20 at one end, with the opposing end releasably attached to a lengthwise disposed strip 49 of hook material on side portion 22, so that the secondary chin strap 47 may be selectively attached, or indeed be left unattached so that 45 piece does not become reduced in size, to generate the exact amount of tension or pressure the user desires for a closely and/or comfortably conforming fit.

Side portion 22, which may be designated the primary treatment or target side portion 22 if the user desires only one gel pack be used for therapy includes an opposing pair of elongated vertically disposed strips 50, 52 of hook or loop type fastening material positioned in spaced parallel relation on outer sheet 32. The strips 50, 52 may extend at least halfway, lengthwise, down the side portion 22 and are sized for releasable substantially overlapping engagement with straps 24, 26. The spacing of the strips 50, 52 corresponds to the width of the placement of straps 24 and 26 so that when standard gel pack 39 is inserted into pocket 38 strips 50, 52 are positioned to apply even pressure to the body region where the wrap 10 is being applied and the lateral edge regions 41 of the gel pack via straps 24, 26. The lateral displacement of straps 24, 26 causes lateral stretching or tension which further conforms the wrap 10 to the contours of the user's face, head or neck. Thus, the positioning of the strips 50, 52 allows for both lateral and lengthwise tensioning of the target side portion 22 of the wrap 10 when straps 24, 26 are attached thereto. Lengthwise tensioning is further aided by the relatively short straps 27, 29 which are attached to straps 24, 26 via buckles 23, 25, the short straps 27, 29 being formed of highly elastic material as previously mentioned. It should be noted that since the straps 27, 29 are short, overtime any elastic or static/non stretch material which may become deformed as might occur with any elastic material, it will not reduce the effectiveness of the tensioning system by straps and attachments 24, 26, 50, 52, 45, 47 and 49. The limited thickness of the gel pack 39 (as opposed to e.g. ice) allows for some deformation thereof, while the inner and outer 30, 32 sheets stretch material has resilience to continue to provide the ability to conform to the contour of the user's face, neck or head. It should be noted that this is accomplished without the use of any laterally extending straps.

It can be seen that straps 24, 26 are elongated to extend nearly the entire length of side pocket 22 to allow for extra-large adult size head, face or neck. This configuration allows for a wide range of adjustability of the wrap 10 while also compressing and deforming the gel pack 39 both laterally and lengthwise relative to how it is being applied (head, and neck as opposed to face) on the target side 22 of the wrap due to the action of straps 24, 26, and the chin strap 44 and tensioning secondary strap 47, respectively, as described above.

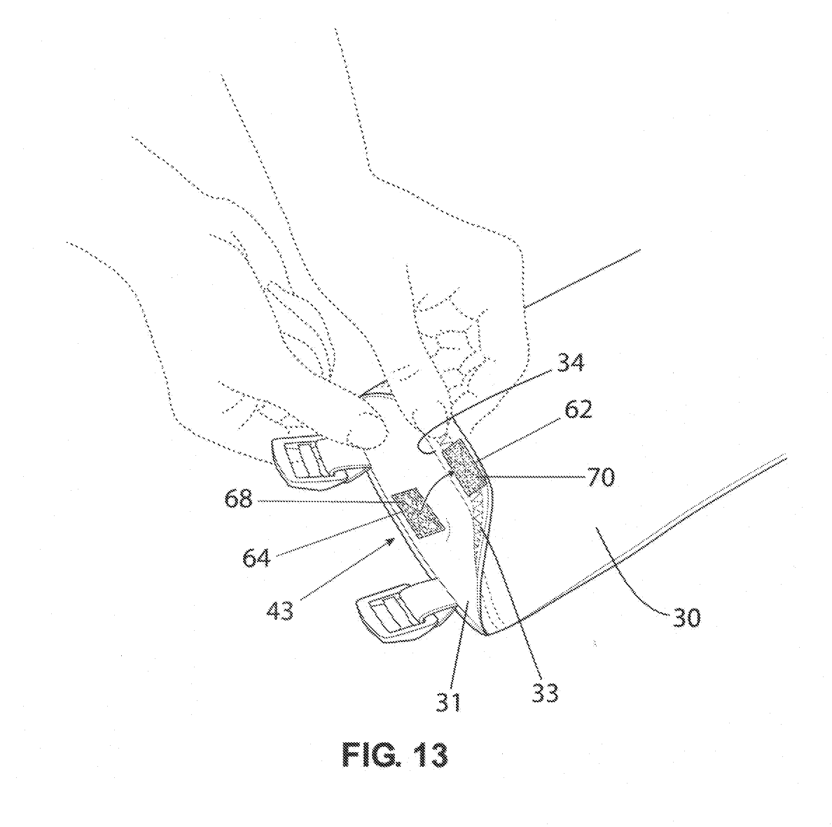

Referring now especially to FIG. 13, it can be seen that each pocket 38 has a closure 43 formed at its top end, the closure formed by cooperating hook and loop fastener patches 62, 64 respectively, positioned on inner 34 and outer 32 sheets respectively. The closure 43 functions to keep the gel pack 39 in position for application to the targeted treatment area while also preventing accidental dislodgement. Both hook and loop fastener tabs 62, 64 are attached via box stitching 68, 70. The “box” stitching that has a diagonal stitch to enhance strength, or alternatively can be secured by an edge stitch around the perimeter of each tab, and reinforced with a stich placed directly over the underlying stitch along the perimeter stitching since repeated detachment of the patches 62, 64, which action places considerable stresses on the central portion of the patches, can cause premature failure of the closure 43. The tabs 62, 64 need to be secured by stitching through sheets 34, 30, and 31, 32 respectively, in order for the structure to retain integrity.

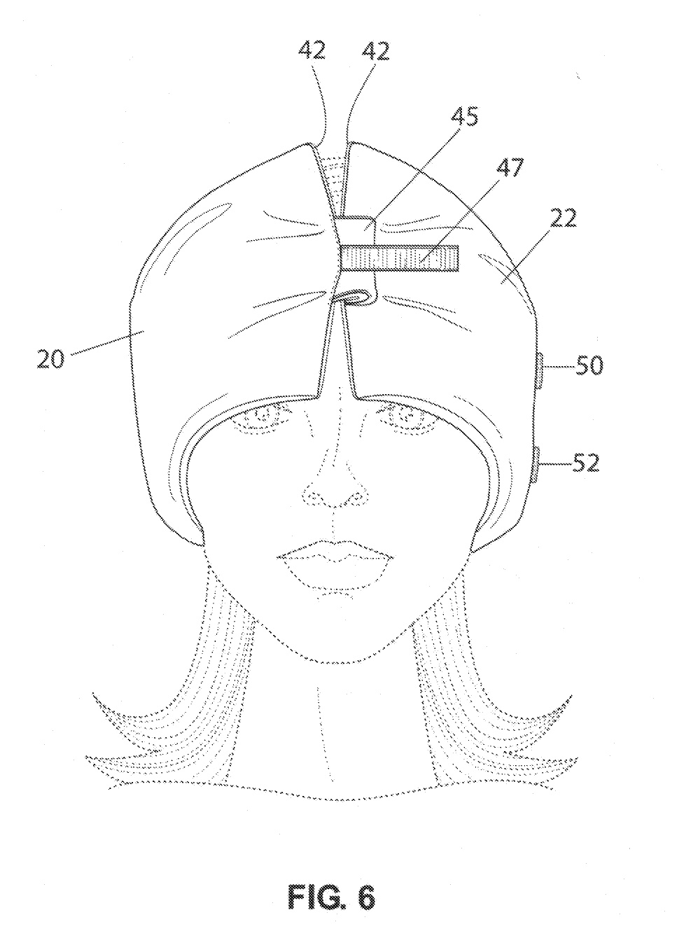

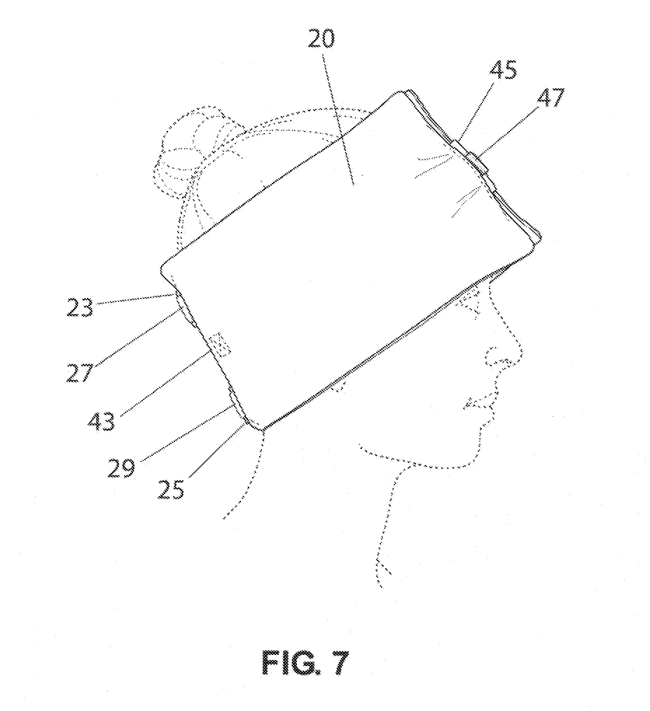

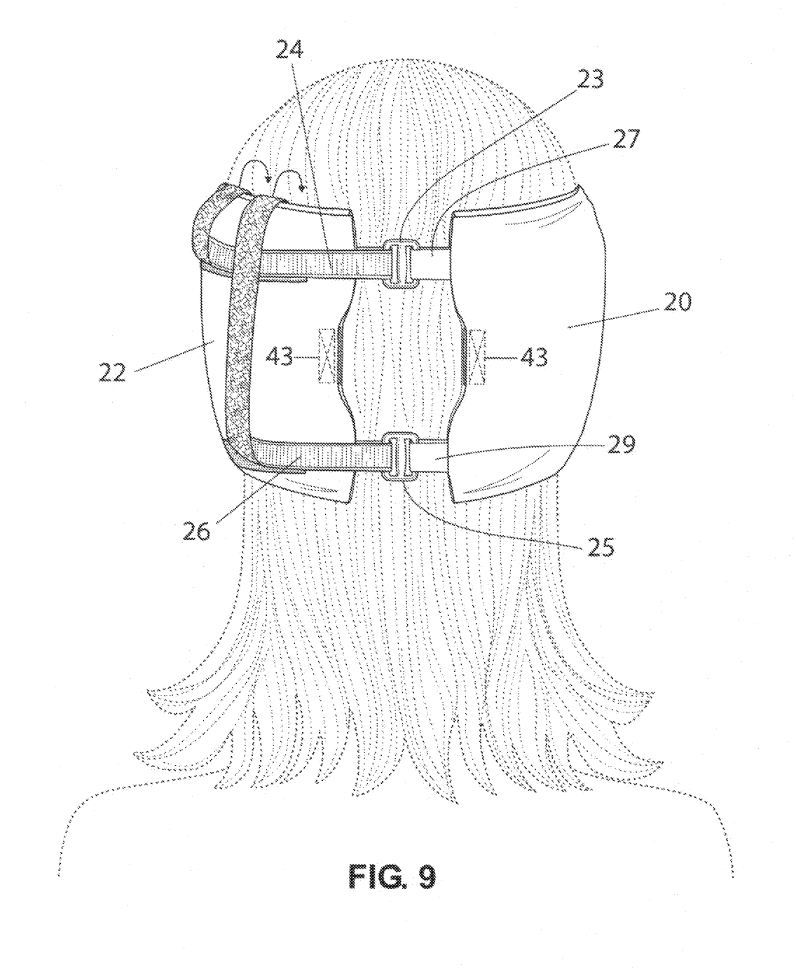

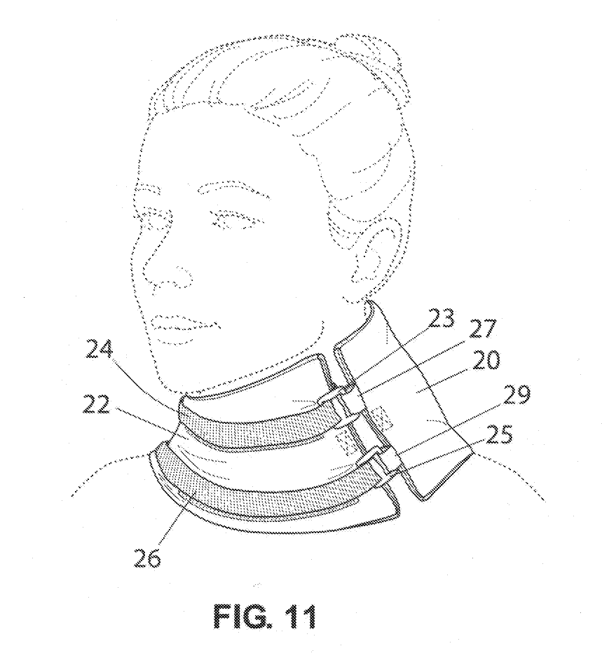

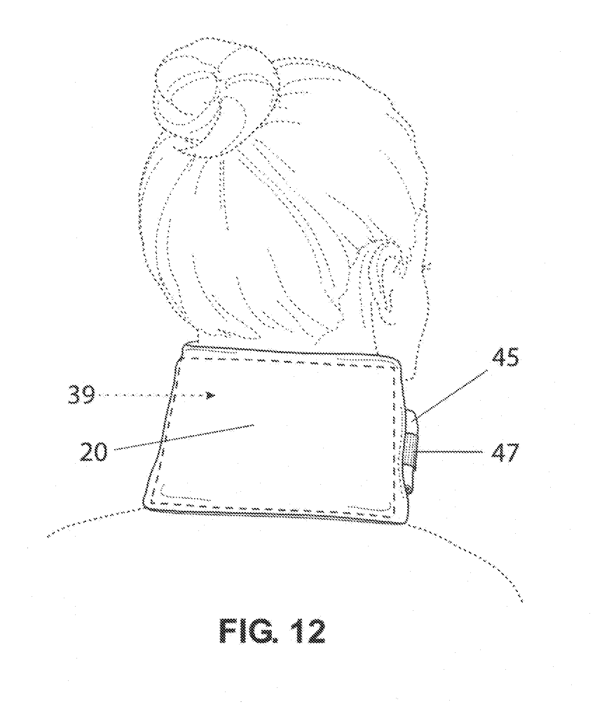

While the wrap 10 is effective for applying heat/cold to the mandibular region of the face, head, or neck, it is equally effective for applying thermal energy to any part of the human body due to the combination of the tensioning of straps 24, 26, going through buckles 23,25, and attaching to pieces 50,52, as well as the adjustable tension system area 45,47, and 49 as well as the resilience of the elastic material. It should be noted that since there is tensioning in three areas of the device, it would not be entirely required to have elasticity in the material being used for the wrap and still achieve an effective tension for contouring to the body. FIGS. 6-12 show the wrap deployed in various positions about the user's head and neck.

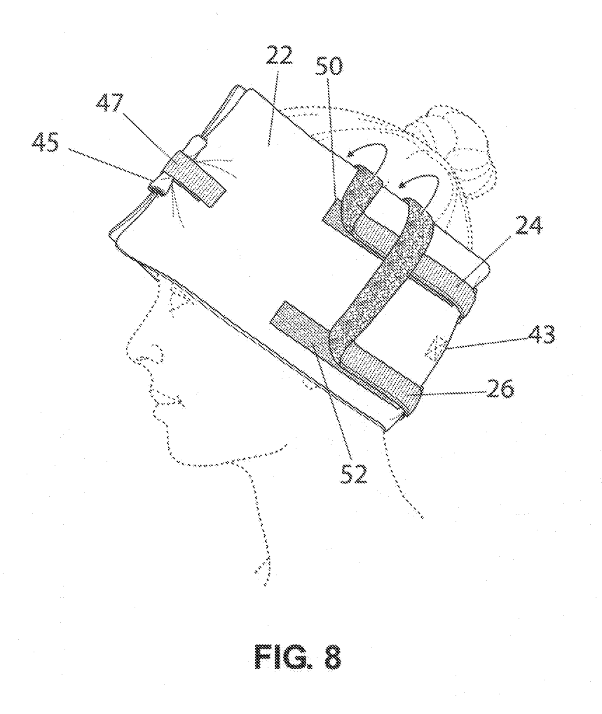

Referring particularly to FIGS. 6-8 the wrap 10 is shown positioned about the crown of the user's head. It should be noted that the wrap can also be positioned so that side portion is placed under the chin while the opposite side portion holds a thermal gel pack to the top of the users head. In this deployment around the forehead and/or crown of the user's head, the wrap 10 is effectively shortened in the length direction by folding over chin strap 45 and using secondary tension strap 47 to pull end portions 42 of the pockets 20, 22 into close proximity. When the portions 20,22 are brought together by strap 47, a more seamless thermal application or continuous thermal coverage of achieved. As the circumference of the wrap 10 is reduced, there is an excess length of straps 24, 26 which may be tucked under the wrap 10 as shown in FIG. 8.

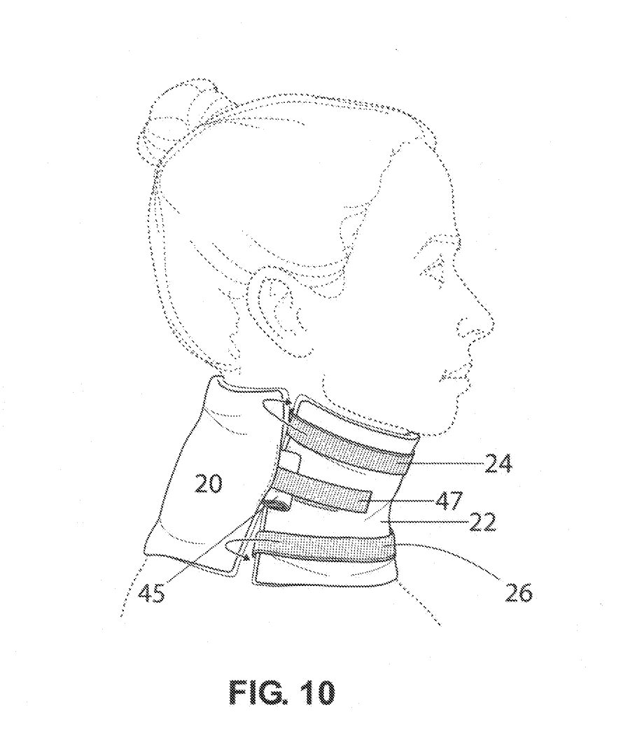

Referring particularly to FIGS. 10-12, the wrap 10 is shown positioned about the user's neck area. The circumference of the wrap is shortened by again folding chin strap 45 with the secondary tension strap 47, and the excess area can be eliminated by additionally deploying straps 24, 26 which attach to 50, 52. The unused ends of the straps once the wrap 10 has been positioned can then be tucked into edge 42 of pocket 22 to prevent dangling thereof, which dangling could interfere with the comfort and safety of the user.

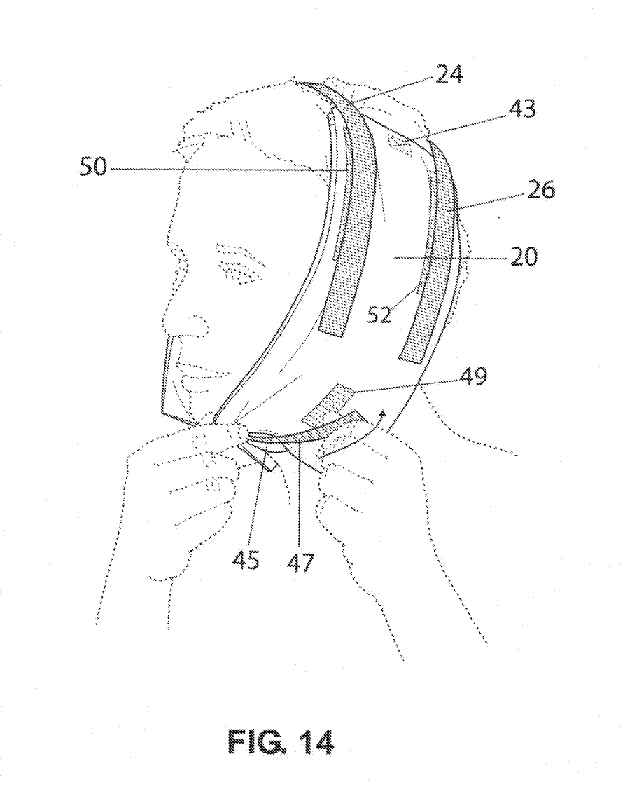

FIG. 14 illustrates deployment of the wrap about the user's mandibular area, cheeks and/or temporomandibular region of the head. After inserting the frozen or heated gel packs 39 into pockets 38 and securing them using closures 43, the user places the wrap 10 about their head and begins the application and tensioning process by inserting straps 24, 26 through their adjacent buckles or tri-bar sliders 23,25, and securing them to respective hook pieces 50,52 on side portion 22. The user can further adjust the tension of the wrap by adjusting the chin piece connecting the two side portions 45 with loop strap 47 and hook fastener 49 according to their desired pressure, comfort, and/or size. The user can also draw the two side portions together so that the pockets meet using strap 47 and piece 49, for a more seamless thermal application.

I claim:

1. A thermal wrap for positioning about a user's head comprising:
said wrap having a pair of opposing side portions, each of said side portions having inner and outer sheets, said side portions selectively connected by at least a pair of horizontally spaced connecting straps, the connecting straps connecting top ends of the side portions;
said wrap including a chin strap or connecting portion between the two side portions pockets, that also includes a permanently attached strap or loop strap portion connected to lower edges of said side portions which connects to a hook fastener on the opposite side portion exterior of the pocket;
each of said side portions including a pocket for receiving a pack of thermal treatment material;
one of said side portions having a pair of co-extensive strips of hook and loop type fastening material positioned on the outer sheet in overlapping relation to said pocket to form a primary pressure and adjustment treatment side of said wrap;
said straps engageable with said strips of hook and loop material to secure the wrap in position about the user's head and secure said primary treatment side in position proximate a target position on said user's face, and also around said user's neck.

2. The wrap of claim 1 wherein said chin strap is approximately one half the width of said side pockets.

3. The wrap of claim 1 wherein the width of said pocket allows for lateral repositioning of said gel pack.

4. The wrap of claim 1 wherein said chin strap portion includes a permanently attached strap, and a releasably attached strap for tensioning.

5. A thermal wrap for positioning about a user's head, face or neck comprising:
said wrap having a pair of opposing side portions, each of said side portions having inner and outer sheets, said side portions selectively connected by at least a pair of horizontally spaced connecting straps, located at the top ends of one side portions that thread through buckles and return to the same side portion and attach to an adjacent hook piece fastener;
said wrap including a chin strap portion made of the same material as the two side portions connected to lower edges of said side portions;
one of said side portions having a pair of co-extensive strips of hook and loop type fastening material positioned on the outer sheet to form a primary treatment side of said wrap;
each of said side portions including a pocket for receiving a pack of thermal treatment material;
said connecting straps engageable with said strips of hook and loop material to secure the wrap in position about the user's head or neck and apply lateral tension to said inner and outer sheets of said primary treatment side of said wrap, said chin strap portion centrally positioned to apply lengthwise tension to said inner and outer sheets of said primary treatment side of said wrap.

6. The wrap of claim of claim 5 wherein said chin strap is about one half the width of said side pockets.

7. The wrap of claim 5 wherein the width of said pocket allows for lateral repositioning of said gel pack.

8. The wrap of claim 5 wherein said chin strap portion includes a permanently attached strap and a releasably attached strap.

9. The wrap of claim 8 wherein said releasably attached strap is about half the width of said permanently attached strap to apply tension in a narrower region of said side portions.

10. A tensioning system for a thermal wrap having two side portions and at least one pocket for receiving thermal treatment material, the system comprising:
a pair of laterally displaced straps attached to a top edge of a first one of said side portions, said at least one pocket positioned on said first one of said side portions, said first side portion having a corresponding pair of laterally displaced attachment strips permanently positioned thereon;
a pair of connectors for adjustably receiving and capturing a desired length of said straps, said connectors attached to a second one of said side portions and laterally displaced to receive said straps;
a chin strap or portion connected at respective bottom edges of said two side portions, said chin strap portion including a permanently attached strap and a releasably attached strap, said releasably attached strap adjustably positionable lengthwise;
whereby tensioning of said wrap is effected by looping the desired length of said straps through said connectors, and securing said straps onto said attachment strips.

11. The wrap of claim 10 wherein said releasably attached strap is about half the width of said permanently attached strap to apply tension in a narrower region of said side portions.

12. The wrap of claim of claim 10 wherein said chin strap portion is about one half the width of said side pockets.

13. The wrap of claim 10 wherein said releasably attached strap is releasably attached to an attachment strip centrally positioned on said first side portion.

14. The wrap of claim 10 wherein said pair of connectors are connected to said second side portion by elasticated material.

15. The wrap of claim 10 wherein said laterally displaced straps and said permanently attached and releasably attached straps of said chin portion are not elasticated.
